(19) United States
(12) Patent Application Publication
(10) Pub. No.: US 2024/0042473 A1
(43) Pub. Date: Feb. 8, 2024

(54) SPRAYING SYSTEM AND SPRAYING MEMBER

(71) Applicant: TECH CORPORATION CO., LTD., Hiroshima (JP)

(72) Inventors: Naoki SATO, Hiroshima (JP); Yoshinori NAKAMOTO, Hiroshima (JP)

(73) Assignee: TECH CORPORATION CO., LTD., Hiroshima (JP)

(21) Appl. No.: 18/265,506

(22) PCT Filed: Dec. 21, 2021
(86) PCT No.: PCT/JP2021/047379

§ 371 Date: Jun. 6, 2023

(30) Foreign Application Priority Data
Dec. 22, 2020 (JP) 2020-212059
Sep. 13, 2021 (JP) 2021-148654

(51) Int. Cl. B05B 12/16; B05B 1/10; B05B 1/20; B05B 7/24

(52) U.S. Cl. CPC B05B 12/16; B05B 1/10; B05B 1/20; B05B 7/2491

(57) ABSTRACT

[Problem] Provided is a spraying system that allows an increase in design freedom.

[Solution] The present invention provides a spraying system comprising: an hypochlorous acid water supplier that supplies hypochlorous acid water; a mist generator that generates mist of the hypochlorous acid water; an upward guiding pipe that guides the mist upward; a lateral guiding pipe connected to the upward guiding pipe and extending in a lateral direction; a downward guiding pipe that is provided below a lower end of the lateral guiding pipe and that guides the mist downward; and a spraying section that is provided downstream from the downward guiding pipe and sprays the mist.

### TECHNICAL FIELD

The present invention is suitably applicable, for example, to a spraying system capable of spraying mist of acidic electrolyzed water onto a spray receiving target object.

### BACKGROUND ART

As a related-art spraying apparatus that sprays hypochlorous acid water having high sterilizing power such as electrolyzed water, there is a known mist spraying apparatus that sprays mist into which electrolyzed water is converted by ultrasonic waves upward (see, for example, Patent Literature 1).

### CITATION LIST

### Patent Literature

Patent Literature 1: JP2015-224856

### SUMMARY OF INVENTION

### Technical Problem

It is conceivable that the mist spraying apparatus described above is applicable to a wider field where hypochlorous acid water mist is used if the apparatus is designed to spray the hypochlorous acid water mist in any direction.

The present invention has been made to solve the problem described above, and an object of the present invention is to provide a spraying system and a spraying member capable of increasing the design flexibility.

### Solution to Problem

To achieve the object described above, a spraying system according to the present invention includes a hypochlorous acid water supplier that supplies hypochlorous acid water,
a mist generator that generates mist of the hypochlorous acid water,
an upward guiding pipe that guides the mist upward,
a lateral guiding pipe connected to the upward guiding pipe and extending in a lateral direction, and
a fan that delivers air to the lateral guiding pipe,
a downward guiding pipe that is provided below a lower end of the lateral guiding pipe and guides the mist downward, and
a spraying section that is provided downstream from the downward guiding pipe and sprays the mist.

A spraying member that sprays hypochlorous acid water according to the present invention includes an upward guiding pipe that guides mist of the hypochlorous acid water upward,
a lateral guiding pipe connected to the upward guiding pipe and extending in a lateral direction,
a fan that delivers air to the lateral guiding pipe,
a downward guiding pipe that is provided below a lower end of the lateral guiding pipe and guides the mist downward, and
a spraying section that is provided downstream from the downward guiding pipe and sprays the mist.

### Advantageous Effects of Invention

The present invention can achieve a spraying system and a spraying member capable of improving the design flexibility.

### BRIEF DESCRIPTION OF DRAWINGS

FIG. 1 is a schematic view showing the configuration of a spraying system according to a first embodiment (front view of spraying apparatus).

FIG. 2 is another schematic view showing the configuration of the spraying system according to the first embodiment (rear view of spraying apparatus).

FIG. 3 is another schematic view showing the configuration of the spraying system according to the first embodiment (right side view of spraying apparatus).

FIG. 4 is a schematic view showing the configuration of a spraying member in the first embodiment.

FIGS. 5A to 5D are schematic views showing a cross-sectional configuration of the spraying member in the first embodiment.

FIGS. 6A and 6B are schematic views for describing the effect of a restrictor in the first embodiment.

FIG. 7 is a schematic view showing the configuration of the restrictor in the first embodiment.

FIG. 8 is a schematic view showing the configuration of the spraying system according to a second embodiment.

FIG. 9 is a schematic view showing the configuration of the spraying system according to another embodiment.

FIG. 10 FIG. 10 is a schematic view showing the configuration of the spraying system according to a third embodiment.

FIGS. 11A and 11B are schematic views showing the configuration of a spray guiding part in the third embodiment.

FIGS. 12A and 12B are schematic views showing the configuration of a spraying port in the third embodiment.

### DESCRIPTION OF EMBODIMENT

Embodiments of the present invention will next be described with reference to the drawings.

### First Embodiment

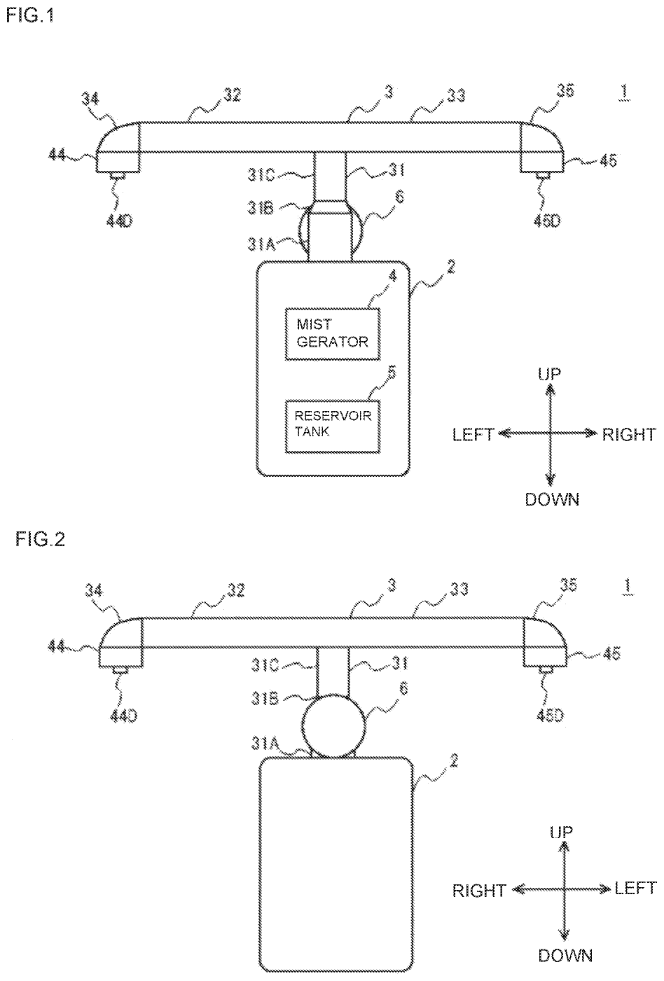

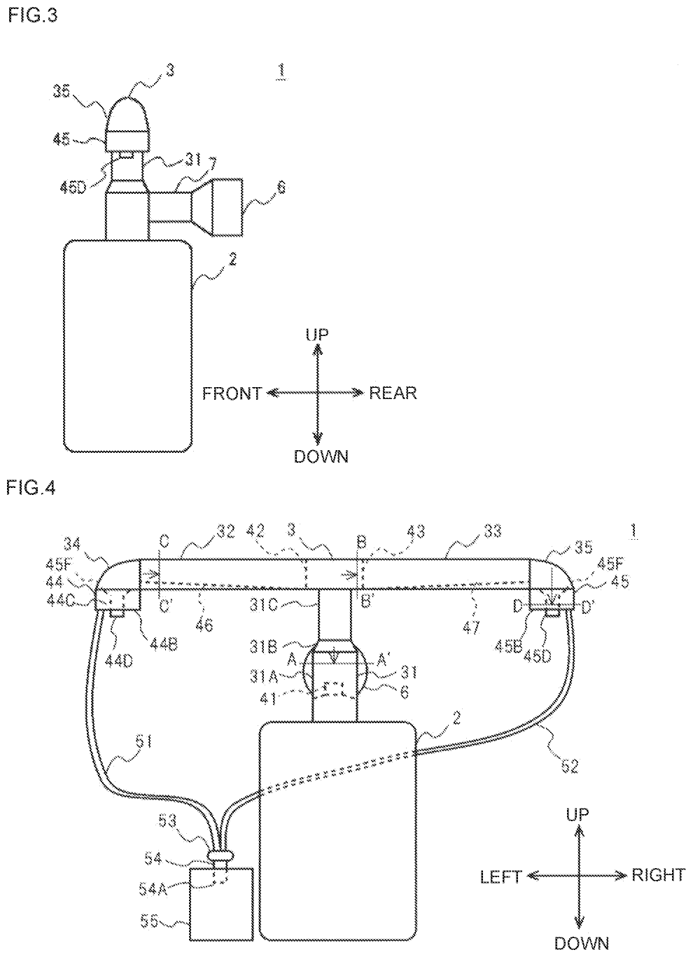

FIG. 1 is a front view of a spraying apparatus 1, FIG. 2 is a rear view of the spraying apparatus 1, and FIG. 3 is a right side view of the spraying apparatus. The spraying apparatus 1 includes an enclosure 2 and a spraying member 3, which is disposed at the top of the enclosure 2, as shown in FIGS. 1 to 3. The spraying apparatus 1 sprays hypochlorous acid water stored in a reservoir tank 5 in the enclosure 2 downward from spraying sections 44 and 45 attached to the spraying member 3. FIGS. 1 to 3 do not show a drain mechanism that will be described with reference to FIG. 4.

Although not shown, the spraying apparatus 1 is so configured that a controller 20 (not shown), which is formed of a micro processing unit (MPU), a read only memory (ROM), and a random access memory (RAM), none of which is shown, oversees and controls the entire spraying apparatus 1.

The enclosure 2 houses the reservoir tank 5 and a mist generator 4. The reservoir tank 5 is a tank that stores the hypochlorous acid water, does not necessarily have a specific configuration, and can be any known tank. For example, a tank having a capacity ranging from 10 to 100 liters, represented by a gallon bottle having a capacity of about 10 liters, is preferably installed. The mist generator 4 and the reservoir tank 5 are omitted from in FIG. 2 and the following figures.

The mist generator 4 is provided so as to be adjacent to the reservoir tank 5, converts the hypochlorous acid water stored in the reservoir tank 5 into mist (atomized fine water droplets), and supplies to the spraying member 3 with the mist. The mist generator 4 does not necessarily generate the mist in a specific method, but preferably uses a method using ultrasonic waves, vaporization, or heated steam.

The hypochlorous acid water stored in the reservoir tank 5 is not necessarily specific water, and can be selected as appropriate from electrolyzed water generated by electrolysis and hypochlorous acid aqueous solution in which sodium hypochlorite is dissolved and the pH adjusted. The electrolyzed water may be acidic electrolyzed water having a pH ranging from 2 to 6.5, neutral electrolyzed water having a pH ranging from 6.5 to 8, or acidic or neutral electrolyzed water diluted with tap water in accordance, and any of the types of electrolyzed water described above is used with the spraying application. For example, acidic electrolyzed water is preferably used for sterilization. Acid electrolyzed water can provide the same disinfection effect at a lower chlorine concentration as sodium hypochlorite water. When acidic electrolyzed water is used, the chlorine concentration thereof preferably ranges from 5 to 50 ppm, particularly from 10 to 30 ppm, to ensure the safety and sterilizing power.

The spraying member 3 includes lateral guiding pipes 32 and 33 extending in the lateral direction and connected to the upper end of an upward guiding pipe 31 extending upward, and has the shape of the letter T as a whole. The phrase “extending upward” may include a state of extending obliquely upward with frontward, rearward, rightward, or leftward inclination with respect to the vertically upward direction by an angle within ±45°. The phrase “extending in the lateral direction” may include a state of extending obliquely in the lateral direction with upward or downward inclination with respect to the horizontal direction by an angle within ±45°.

The upward guiding pipe 31 has a large-diameter section 31A, a relay section 31B, and a small-diameter section 31C arranged from below or the upstream side toward above or the downstream side. The large-diameter section 31A is larger in diameter than the small-diameter section 31C, and the two sections are connected to each other via the relay section 31B, the diameter of which decreases from below toward above.

The lateral guiding pipes 32 and 33, which extend in the lateral direction, are connected to the upper end of the upward guiding pipe 31. In FIGS. 1 and 2, the lateral guiding pipes 32 and 33 extend in a rightward-leftward direction, and may instead extend in a frontward-rearward direction.

Downward guiding pipes 34 and 35 are connected to the left and right ends of the lateral guiding pipes 32 and 33, respectively. The downward guiding pipes 34 and 35 change the piping direction to the downward direction. The spraying sections 44 and 45, via which the mist is sprayed, are formed at the lower terminal ends of the downward guiding pipes 34 and 35.

A fan pipe 7 connected to a fan 6, which delivers air, is connected to a portion near the downward terminal end of the upward guiding pipe 31. The portion near the downward terminal end means that the lower end of the fan pipe 7 is located at 20 cm or shorter from the upstream terminal end of the upward guiding pipe 31.

The mist supplied from the mist generator 4 to the upward guiding pipe 31 is therefore urged by the airflow from the fan 6, delivered along with the airflow from the fan 6 to the lateral guiding pipes 32 and 33, and sprayed downward from the spraying sections 44 and 45 through the downward guiding pipes 34 and 35. The urging effect provided by the fan 6 causes the mist to be promptly sprayed via spraying ports 44D and 45D. A lower blocker (not shown, provided with no gap forming section) having the shape of the letter C, which is similar to lower blocking sections 42 and 43, which will be described later, is provided upstream from the fan 6 (rearward in FIGS. 2 and 3) and suppresses travel of the water droplets toward the rear side of the fan 6. A mesh may be installed behind the fan 6 to prevent airflow dust contamination.

The mist (fine water droplets) is characterized so as to gradually increase in size as the mist particles repeatedly collide with each other into droplets, which then fall downward. If the mist is sprayed upward, the water droplets are allowed to travel directly through a pipe and return to the reservoir tank. When the mist is moved laterally and downward, however, water droplets are generated as the mist moves, and undesirably accumulate inside the lateral guiding pipe or drip down from the spraying sections.

To avoid the problems described above, in the invention of the present application, the amount of water droplets that fall from the spraying section 45 is reduced by suppressing the generation of the water droplets or by causing the water droplets to return to the reservoir tank 5.

FIG. 4 shows the internal structure of the spraying member 3 with dashed lines and further shows the drain mechanism. FIG. 5A shows the cross section taken along the line A-A′ in FIG. 4. FIG. 5B shows the cross section taken along the line B-B′ in FIG. 4. FIG. 5C shows the cross section taken along the line C-C′in FIG. 4. FIG. 5D shows the cross section taken along the line D-D′ in FIG. 4.

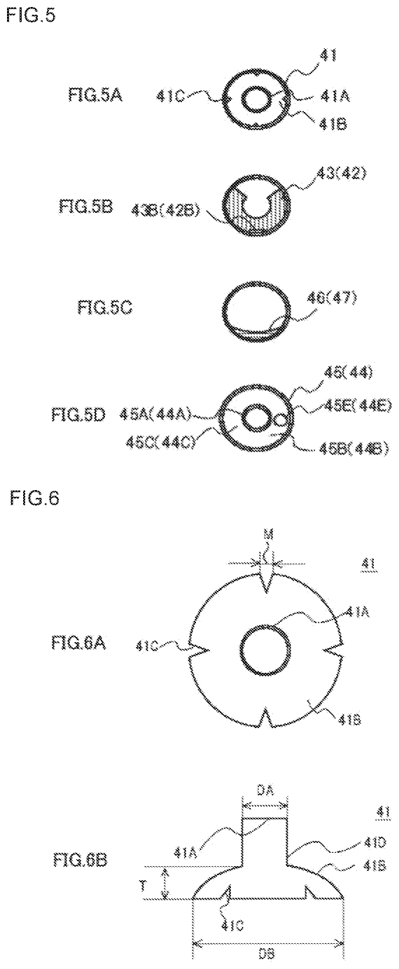

A restrictor 41 is disposed near the lower end of the large-diameter section 31A (within 10 cm from lower end), as shown in FIGS. 4 and 5A to 5D. The restrictor 41 includes a cylindrical section 41D, a central portion of which has restricting hole 41A having an inner diameter smaller than the small-diameter section 31C (preferably 70 to 30% of small-diameter section 31C), a peripheral blocking section 41B, which extends outward from the restricting hole 41A and slightly (by 3 to 20°) inclines downward, and a plurality of (four in FIG. 6A) circumferential edge holes 41C formed at the circumferential edge of the peripheral blocking section 41B, as shown in FIGS. 6A and 6B. For example, the restrictor 41 is so designed that an inner diameter DA of the restricting hole 41A is 16 mm, the inner diameter of the large-diameter section 31A and an outer diameter DB of the peripheral blocking section 41B are 60 mm, and a thickness T of the peripheral blocking section 41B is 15 mm.

The mist traveling upward is therefore partly reflected off the peripheral blocking section 41B and returns to the reservoir tank 5, while the mist in the central portion is sprayed with great force via the restricting hole 41A. Out of the mist located near the circumferential edge and has a high collision probability and the mist reflected off the peripheral blocking section 41B, heavy mist cannot pass through the restricting hole 41A, while small mist can selectively pass through the restricting hole 41A. The water droplets traveling downward from the downstream side are allowed to return to the reservoir tank 5 through the circumferential edge holes 41C. The shape or size of the circumferential edge holes 41C are not limited to a specific shape or size. For example, the circumferential edge holes 41C are each formed as a notch having bases of a triangular shape.

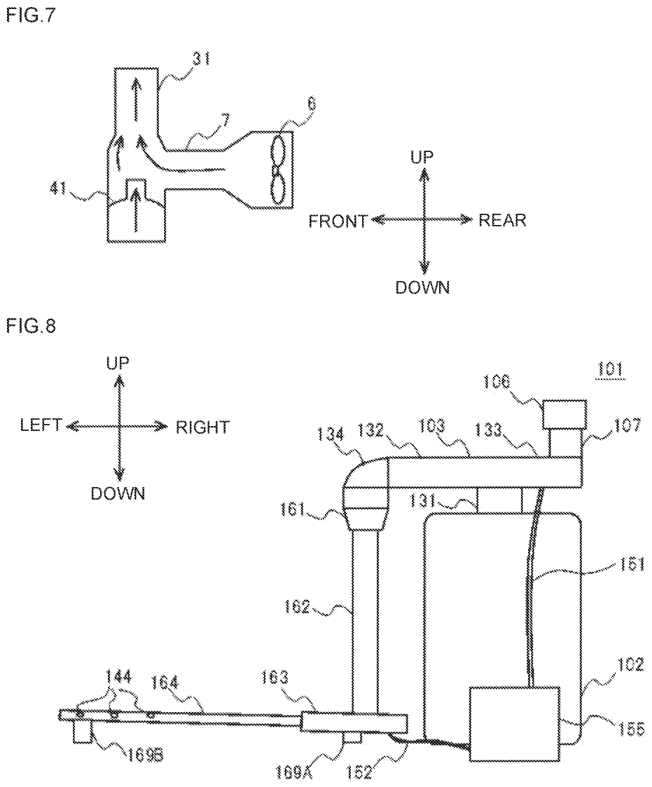

The restrictor 41 raises the mist via the restricting hole 41A and deflects the direction of the airflow delivered from the fan 6 from the forward direction to the upward direction, as shown in FIG. 7. At this point of time, the mist is sprayed with great force via the restricting hole 41A, which has a small diameter, and can therefore travel upward without being pushed back. The cylindrical section 41D, which protrudes from the peripheral blocking section 41B, can create turbulence in the traveling direction of the airflow and cause the mist to rise mixed with the airflow.

The lower blocking sections 42 and 43 are disposed near (within 20 cm from connected portion in rightward-leftward direction described below) the portion where the upward guiding pipe 31 branches into the lateral guiding pipes 32 and 33 (where lateral guiding pipes 32 and 33 are connected to upward guiding pipe 31). The lower blocking sections 42 and 43 each have the shape of the letter C having a notch that opens upward over an angle ranging from 60 to 150° (about 90° in FIG. 5B) based on a donut shape having an outer diameter approximately equal to that of the lateral guiding pipes 32 and 33 and a hollowed-out central portion. The diameter of the central portion is 70 to 30% of the inner diameter of the lateral guiding pipes 32 and 33. Gap forming sections 42B and 43B, which form gaps between the lower blocking sections 42, 43 and the lateral guiding pipes 32, 33, are formed on the lower side of the lower blocking sections 42 and 43.

The lower blocking sections 42 and 43 selectively allow the mist that is light in weight and located on the upper side and the mist near the central portion, where the collision probability is small, to pass therethrough, while allowing the water droplets traveling from the downstream side to return via the gap forming sections 42B and 43B to the reservoir tank 5.

The lateral guiding pipes 32 and 33 include inclining sections 46 and 47, which incline downward from the downstream side toward the upstream side. The inclining sections 46 and 47 can guide the water droplets traveling from the downstream side toward the upstream side and cause the water droplets to return to the reservoir tank 5 via the gap forming sections 42B and 43B. The gap forming sections 42B and 43B are provided only when the inclining sections 46 and 47 are disposed. That is, when the inclining sections 46 and 47 are not disposed, lower portions of the lower blocking sections 42 and 43 are provided in contact with the lateral guiding pipes 32 and 33.

The downward guiding pipes 34 and 35, which change the direction of the mist to the downward direction, are formed at the left and right terminal ends (downstream ends) of the lateral guiding pipes 32 and 33. The downward guiding pipes 34 and 35 each have a gently curved outer portion, which changes the direction of the mist to the downward direction along the smoothly curved line. The spraying sections 44 and 45 are connected to the lower ends of the downward guiding pipes 34 and 35.

The spraying sections 44 and 45 include central cylinders 44A and 45A, which are open upward and downward, and guiding sections 44F and 45F, which spread upward from the central cylinders 44A and 45A. There is a difference in position between bottom surfaces 44B, 45B formed at the exterior of the central cylinders 44A, 45A and the top ends of the central cylinders 44A, 45A. In other words, the front ends of funnel shapes having inverted conical body sections (guiding sections 44E and 45E) around holes formed in the bottom surfaces 44B and 45B, and the central cylinders 44A and 45A, which form legs extending from the front ends of the conical shapes, are disposed as the spraying ports 44D and 45D.

Water droplet storages 44C and 45C according to the differences between the bottom surfaces 44B, 45B and the top ends of the central cylinders 44A, 45A are thus formed. The water droplets generated downstream from the downward guiding pipes 34 and 35 can be stored in the water droplet storages 44C and 45C, which can suppress fall of the water droplets via the spraying ports 44D and 45D, which are the downward opening of the central cylinders 44A and 45A.

The water droplet storages 44C and 45C are each provided with the drain mechanism, which discharge the stored water droplets. Bottom surface holes 44E and 45E are provided in the bottom surfaces 44B and 45B of the water droplet storages 44C and 45C, and drainpipes 51 and 52 are connected to the bottom surface holes 44E and 45E, respectively. The drainpipes 51 and 52 are linked to each other via a linkage 53, and the water droplets are discharged via a discharge port 54A of a common discharge pipe 54. The front end of the common discharge pipe 54 is inserted into a drain tank 55, and the water droplets are stored in the drain tank 55.

The drain mechanisms (bottom surface holes 44E and 45E, drainpipes 51 and 52, linkage 53, common discharge pipe 54, and drain tank 55) may be omitted. In this case, for example, the spraying sections 44 and 45 are fitted to the downward guiding pipes 34 and 35 in a removable manner with screws or other fasteners, and a configuration in which only the spraying sections 44 and 45 are readily removed from the downward guiding pipes 34 and 35 allows the hypochlorous acid water stored in the water droplet storages 44C and 45C to be readily discarded.

### Second Embodiment

A second embodiment will next be described with reference to FIG. 8. The second embodiment differs from the first embodiment in that the hypochlorous acid water is sprayed upward from spraying sections 144. Portions corresponding to those in the first embodiment have reference characters of the corresponding portions to which 100 is added, and the same portions will not be described.

In a spraying apparatus 101, a lateral guiding pipe 132, which extends in the rightward-left direction, is connected to an upward guiding pipe 131, which has a length ranging from about 10 to 15 cm. Although not shown, the restrictor 41 is disposed (see FIG. 6) in the upward guiding pipe 131, as in the first embodiment. A drainpipe 151 is connected to a portion of the lateral guiding pipe 133 that is a portion near the right root of the lateral guiding pipe 133 (root facing upward guiding pipe 131) where no downward guiding pipe 134 is provided.

A fan 106 and an airflow pipe 107 are connected to a front-end-side portion of the lateral guiding pipe 133, and air is delivered from above to below. The configuration described above reduces the number of droplets that drip outward from the upstream side (rightward) of the fan 106.

The spraying apparatus 101 is provided with a vertical pipe 162, which extends downward from the downward guiding pipe 134, a first parallel pipe 163, which is disposed in parallel to the floor surface (within ±100 with respect to floor surface), and a second parallel pipe 164, which further extends in parallel to the floor surface. The spraying sections 144, which are a plurality of holes, are formed in a front end (left) portion of the second parallel pipe 164.

The spraying sections 144, with which the second parallel pipe 164 are provided, are provided so as, for example, to face in the upward direction or incline by a predetermined spraying angle with respect to the upward direction. For example, when two spraying sections 144 having a spraying angle of 45° are provided in the frontward-rearward direction, the hypochlorous acid water can be sprayed obliquely upward over a wide angle. Instead, when two spraying sections 144 having a spraying angle of 90° are provided in the frontward-rearward direction, the hypochlorous acid water can be sprayed approximately in parallel to the floor surface.

The spraying sections 144 may be provided in the form of a plurality of spraying sections 144 or a plurality sets of spraying sections 144 in the longitudinal direction. In this case, it is preferable to provide the spraying sections 144 separate from each other by a distance ranging, for example, from 20 to 100 cm in the longitudinal direction. The size or number of spraying sections 144 is not limited to a specific size or number, and is selected as appropriate in accordance with the output and other factors of the spraying apparatus 101. It is preferable to provide 4 to 20 spraying sections 144 each having a diameter ranging 4 to 10 mm. For example, two spraying sections 144 each having a diameter of about 5 mm are arranged in the frontward-rearward direction, and three rows of the two spraying sections 144 can be provided. Six spraying sections 144 in total can thus be provided.

The first parallel pipe 163 and the second parallel pipe 164 are fixed to the floor surface via installation fixtures 169A and 169B. The installation fixture 169B is taller than the installation fixture 169A, so that the parallel pipes as a whole slightly incline downward (within 10°) along the direction from the front end of the second parallel pipe 164 toward the root of the first parallel pipe 163, that is, toward the upstream side. A drainpipe 152 is connected to a right portion of the first parallel pipe 163, and the droplets generated in the first parallel pipe 163 and the second parallel pipe 164 are collected through the drainpipe 152. The root end of the first parallel pipe 163 may be connected to an enclosure 102 to cause the droplets to return to a reservoir tank 104 (not shown) or store the wastewater.

In the spraying apparatus 101, the diameters (that is, cross-sectional areas) of the following pipes decrease toward the downstream side: the upward guiding pipe 131; the lateral guiding pipe 132; the downward guiding pipe 134; the vertical pipe 162; the first parallel pipe 163; and the second parallel pipe 164. The configuration described above allows the decrease in the number of mist particles, which are converted into droplets, to be cancelled by a pressure increase associated with the decrease in the cross-sectional area of the mist particles, whereby the force of the mist sprayed via the spraying sections 144 can be maintained. For example, the diameter of the upward guiding pipe 131 is 7.6 cm, whereas the diameter of the second parallel pipe 164 is 1.6 cm. The ratio of the diameter of the upward guiding pipe 131 to the diameter of the second parallel pipe 164 preferably ranges from about 20:1 to 2:1, more preferably from about 15:1 to 3:1. The first parallel pipe 163 and the second parallel pipe 164 are set to have a total length ranging from about 1 to 10 m, preferably from about 2 to 8 m.

### Operation and Effects in First and Second Embodiments

The features of the group of inventions extracted from the embodiments described above will be described below with problems, effects, and other factors presented as necessary. In the following description, for ease of understanding, corresponding configurations in the embodiments described above are presented, for example, in parentheses as appropriate, but are not limited to the specific configurations presented, for example, in parentheses. The meanings, examples, and other attributes of terms described in each feature may be applied as the meanings and examples of terms described in other features described in the same wording.

According to the configurations described above, a spraying system (spraying apparatus 1) according to the present invention includes
a hypochlorous acid water supplier (reservoir tank 5) that supplies hypochlorous acid water,
a mist generator (mist generator 4) that generates mist of the hypochlorous acid water,
an upward guiding pipe (upward guiding pipe 31) that guides the mist upward,
a lateral guiding pipe (lateral guiding pipe 32 or 33) connected to the upward guiding pipe and extending in the lateral direction,
a fan (fan 6) that delivers air to the lateral guiding pipe,
a downward guiding pipe (downward guiding pipes 34 and 35) that is provided below the lower end of the lateral guiding pipe and guides the mist downward, and
a spraying section (spraying sections 44 and 45) that is provided downstream from the downward guiding pipe and sprays the mist.

The spraying system can thus spray the mist of the hypochlorous acid water, which travels upward, downward due to gravity, thereby preventing a user from accidentally inhaling a large amount of hypochlorous acid water and therefore enhancing the safety of the user. The hypochlorous acid water can also be sprayed intensively onto the lower body of the user, such as the sites under the feet. To increase the safety, the spraying section is preferably located at a height of 130 cm or shorter, more preferably 100 cm or shorter from the floor surface.

In the spraying system, the spraying section is formed of a plurality of spraying sections.

The spraying system can thus spray the hypochlorous acid water downward from the plurality of locations, whereby the design flexibility can be improved.

In the spraying system, the spraying sections each include a water droplet storage that can store water droplets around a spraying port.

The spraying system can thus keep the water droplets in the spraying section and suppress drip of the water droplets via the spraying section when the mist is sprayed downward.

In the spraying system, the hypochlorous acid water supplier is a tank that stores the hypochlorous acid water.

The spraying system can thus spray the hypochlorous acid water through the simple configuration.

In the spraying system, the hypochlorous acid water supplier is an electrolysis section that electrolyzes a supplied electrolyte solution and raw water.

The spraying system can thus generate the hypochlorous acid water through electrolysis, eliminating the need to separately prepare the hypochlorous acid water.

In the spraying system, the upward guiding pipe includes an upstream section having a large diameter, a downstream section having a small diameter, and a connector that connects the upstream and downstream sections to each other.

In the spraying system, the diameter of the upward guiding pipe can therefore be changed from the large diameter to the small diameter, whereby the mist can be raised with great force.

In the spraying system, a restrictor that reduces the inner diameter of the upward guiding pipe is formed in the upward guiding pipe.

The spraying system can thus selectively raise the central portion of the mist.

In the spraying system, the restrictor includes
a restricting hole formed in a center portion,
a peripheral blocking section that extends from the restricting hole toward the periphery, inclines downward, and does not allow the mist around the restricting hole to pass therethrough,
a circumferential edge hole formed at the circumferential edge of the peripheral blocking section formed inside of the upward guiding pipe.

The spraying system can thus selectively raise the central portion of the mist and cause heavy water droplets to return from the circumferential edge hole to the hypochlorous acid water supplier.

In the spraying system, a lower blocking section that blocks the mist that is about to pass through the lower portion of the lateral guiding pipe is provided in the lateral guiding pipe.

The spraying system can thus cause the mist having small particle sizes and located in the central portion and upper side of the lateral guiding pipe to selectively pass through the lateral guiding pipe, thereby suppressing generation of water droplets in the downstream stages.

In the spraying system, an inclination section that inclines from the downstream side toward the upstream side is provided in the lateral guiding pipe.

The spraying system can thus cause the water droplets generated in the lateral guiding pipe to return to the hypochlorous acid water supplier.

A spraying member according to the present invention includes an upward guiding pipe that guides mist of hypochlorous acid water upward, a lateral guiding pipe connected to the upward guiding pipe and extending in the lateral direction, a spraying section that is provided on the lower side of the lateral guiding pipe and sprays the mist downward, and a fan that is connected to the upward guiding pipe and delivers air into the upward guiding pipe.

The spraying member can thus spray the hypochlorous acid water downward by using the spraying apparatus typically having a spraying port on the upper side.

### Third Embodiment

A third embodiment will next be described with reference to FIGS. 10, 11A, 11B, 12A, and 12B. The third embodiment differs from the first embodiment in terms of the shape of the spraying member. Portions corresponding to those in the first embodiment have reference characters of the corresponding portions to which 200 is added, and the same portions will not be described.

In a spraying apparatus 201 according to the present embodiment, the height of an enclosure 202 is about 40 cm, the distance from the floor to the upper end of the spraying apparatus 201 is about 100 cm, and a spraying member 203 is provided with no fan, as shown in FIG. 10.

A reservoir tank 205 and a mist generator 204 are integrated with each other, and the mist generator 204 is provided at the bottom surface of a reservoir tank 205. A fixed hole 202A is formed in the upper surface of the enclosure 202, and an upward guiding pipe 231 is inserted through the fixed hole 202A and fixed to the enclosure 202.

After the spraying member 203 inserted into the reservoir tank 205 via the upper surface of the enclosure 202 is removed, the upper surface (not shown) of the enclosure 202 is removed to cause part of entirety of the upper surface of the reservoir tank 205 to form an opening, and the reservoir tank 205 is replenished with electrolyzed water via the opening.

A mist path 204A is connected to the top of the mist generator 204, and the top of the mist path 204A is connected to the upward guiding pipe 231. The mist generated by the mist generator 204 is carried by the airflow produced by a fan disposed in the mist generator 204, and supplied to the upward guiding pipe 231 through the mist path 204A.

A restrictor 241 (see FIG. 10) is disposed at the top of the mist path 204A. The restrictor 241 has substantially the same configuration as that of the restrictor 41 in the first embodiment. For example, the restrictor 241 is formed as follows: The outer diameter is 70 mm; the diameter of a restricting hole 241A ranges from 20 to 50 mm (about ¼ to ¾ of outer diameter); the radial length of each circumferential edge hole 241C is 7 mm; and the circumferential width of each circumferential edge hole 241C is 8 mm.

In other words, the restrictor 241 covers the upper end of the mist path 204A, and the restricting hole 241A serves as the discharge port of the mist path 204A, so that the outlet via which the mist is directed toward the upward guiding pipe 231 is narrowed. The upper end of the restricting hole 241A is located at roughly the same position as the position of a lower end 231K of the upward guiding pipe 231, and the mist sprayed via the restricting hole 241A is guided to the upward guiding pipe 231.

The mist introduced into the upward guiding pipe 231 travels upward. A spray guiding part 249 is disposed at the top (portion near upper end) of the upward guiding pipe 231, that is, immediately before a portion where the upward guiding pipe 231 is connected to lateral guiding pipes 232 and 233. The portion near the upper end of the upward guiding pipe 231 means that the upper end of the spray guiding part 249 is located at 10 cm or shorter from the lower end of the connection portion.

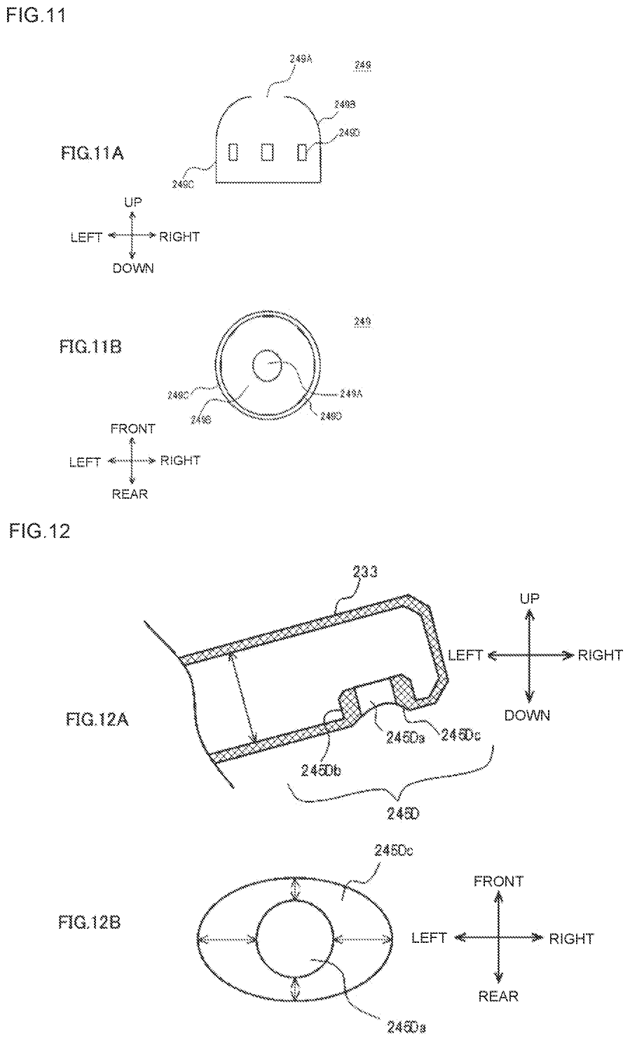

The spray guiding part 249 has the shape of a dome as a whole and includes an upper opening 249A, which is located at the center of the top surface, a curved section 249B, the diameter of which decreased in the upward direction, and a cylindrical section 249C, which has the shape of a cylinder, as shown in FIGS. 11A and 11B.

The spray guiding part 249 has eight rectangular drain holes 249D formed at the boundary in the upward-downward direction between the curved section 249B and the cylindrical section 249C.

The sizes of the portions of the spray guiding part 249 are not limited to specific sizes, and it is desirable to design the portions as appropriate in accordance with the outer diameter of the upward guiding pipe 231. For example, the overall diameter of the spray guiding part 249 is 70 mm, the diameter of the opening 249A is 25 mm, the lower end of each of the drain holes 249 is located at a position of 10 mm from the lower end of the spray guiding part 249, and the drain holes 249 each have a vertical size of 10 mm and a horizontal size of 4 mm.

The spray guiding part 249 allows the central mist to pass therethrough and the water droplets that fall via the drain holes 249 to guide downward, as the restrictor 241 does. The diameter of the curved section 249B gently decreases as compared with the restrictor 241, so that a greater amount of mist is guided upward by the spray guiding part 249 than the mist guided by the restrictor 241.

In other words, in the restrictor 241, in which the diameter is steeply reduced and the area of the discharge port is limited, the mist is sprayed with great force via the restricting hole 241A, and sprayed upward because the restricting hole 241A has a certain length in the height direction. In contrast, in the spray guiding part 249, the mist guided along the curved section 249B is sprayed via the opening 249A, but the opening 249A does not have a length in the height direction, so that the mist spreads in the rightward-leftward direction and is guided by itself to the lateral guiding pipes 232 and 233.

The lateral guiding pipes 232 and 233 are connected to the upward guiding pipe 231 with the lateral guiding pipes 232 and 233 lowering toward the center of the spraying member 203 (upward guiding pipe 231), for example, inclining with respect to the lateral direction by about 15°. That is, the mist having passed through the upward guiding pipe 231 is guided to the two lateral guiding pipes 232 and 233, into which the upward guiding pipe 231 branches slightly upward.

The inclining lateral guiding pipes 232 and 233 cause the water droplets generated in the lateral guiding pipes 232 and 233 to return to the reservoir tank 205 via the upward guiding pipe 231.

Small irregularities (surface roughness Ra ranging from 1 to 100 μm) are formed at the inner surface (not shown) of each of the lateral guiding pipes 232 and 233. The irregularities serves as a portion that collects the mist having increased particle diameters into large droplets.

Spraying ports 244D and 245D are formed near the ends of the lateral guiding pipes 232 and 233. The phrase “near the ends of the lateral guiding pipes 232 and 233” means that the outer ends of the spraying ports 244D and 245D are each located at a distance of ⅕ or shorter, furthermore 1/10 or shorter, of the length of the lateral guiding pipes 232 and 233. The spraying port 245D will be described below, and the spraying port 244D has the same configuration as that of the spraying port 245D.

The spraying port 245D has a wall section 245Db having an elliptical columnar shape and protruding inward from the bottom surface of the lateral guiding pipe 233, and a hole 245Da is formed at the center of the wall section 245Db, as shown in FIGS. 12A and 12B. The height of the wall section 245Db is greater than the height of the inner surface of the lateral guiding pipe 233 at the front and rear ends of the hole 245Da.

The water droplets generated at the right side of the hole 245Da in the lateral guiding pipe 233 are therefore guided leftward along the front and rear sides of the wall section 245Db, and returns into the reservoir tank 205 via the upward guiding pipe 231.

An elliptical recess 245Dc, which is smoothly recessed inward from the bottom surface of the lateral guiding pipe 233, is formed outside the spraying port 245D. The mist is thus sprayed via the hole 245Da along the shape of the recess 245Da with the mist greatly diffused in the horizontal direction.

### Operation and Effects in Third Embodiment

The features of the group of inventions extracted from the embodiments described above will be described below with problems, effects, and other factors presented as necessary. In the following description, for ease of understanding, corresponding configurations in the embodiments described above are presented, for example, in parentheses as appropriate, but are not limited to the specific configurations presented, for example, in parentheses. The meanings, examples, and other attributes of terms described in each feature may be applied as the meanings and examples of terms described in other features described in the same wording.

A spraying system according to the present invention (spraying apparatus 201) includes a hypochlorous acid water supplier (reservoir tank 205) that supplies hypochlorous acid water,
a mist generator (mist generator 204) that generates mist of the hypochlorous acid water,
an upward guiding pipe (upward guiding pipe 231) that guides the mist upward,
a lateral guiding pipe (lateral guiding pipe 232 or 233) connected to the upward guiding pipe and extending in the lateral direction, and
a spraying section (spraying port 245D) which is located near the end of the lateral guiding pipe and via which the mist is sprayed downward.

The spraying system can thus spray the mist downward, thereby preventing the user from inhaling a large amount of mist as much as possible and therefore enhancing the safety of the user.

In the spraying system, the lateral guiding pipe extends outward and inclines upward by an angle ranging from 1 to 45°, and
the spray section is provided downward
at a portion having ⅓ of the length of the lateral guiding pipe from the end of the lateral guiding pipe. The spray section may instead be located at a distance of ⅓ or shorter of the length of the lateral guiding pipe from the end of the lateral guiding pipe and provided so as to face in the downward direction.

The water droplets generated in the lateral guiding pipe can thus be guided so as to return into the upward guiding pipe along the inclination of the lateral guiding pipe.

In the spraying system, the spraying section is a spraying hole provided in the lateral guiding pipe, and a protrusion (wall section 245Db) that protrudes toward the interior of the lateral guiding pipe is present around the circumferential edge of the spraying hole.

The configuration described above allows the water droplets generated on the upper side of the spraying section in the lateral guiding pipe to pass by the side surface of the protrusion and return into the upward guiding pipe, preventing the water droplets from dripping down via the spraying section.

### Other Embodiments

In one of the embodiments described above, the reservoir tank 5 is used as the hypochlorous acid water supplier, but not necessarily in the present invention. For example, the reservoir tank 5 may instead be an electrolysis section including a one-chamber-type electrolytic cell, a two-chamber-type electrolytic cell including an anode chamber having an anode and a cathode chamber having a cathode, or a three-chamber-type electrolytic cell including an intermediate chamber to which an electrolyte aqueous solution is supplied in addition to the anode chamber and the cathode chamber.

In the embodiments described above, electrolyzed water generated by electrolysis is sprayed, but not necessarily in the present invention, and bubbled electrolyzed water containing fine bubbles may instead be sprayed. The bubbled electrolyzed water is electrolyzed water containing nano-order-sized (1 to 999 nm) nanobubbles the number of which is greater than or equal to the seventh power of 10 per milliliter produced by a physical impact effect (such as high-speed swirl, pressure release, Venturi, or a combination thereof) without using chemical treatment agents before or after electrolysis.

In one of the embodiments described above, the spraying section 45 having the spraying port 45D is disposed below the downward guiding pipe 35, but not necessarily in the present invention. For example, the spraying port may be disposed on the lower side of a portion near the terminal end of the lateral guiding pipe. The restrictor 41, the lower blocking sections 42 and 43, the inclining sections 46 and 47, the water droplet storages 44C and 45C, and the drain mechanisms are not essential and are selectively used as appropriate. The configurations of the restrictor 41, the lower blocking sections 42 and 43, and the inclining sections 46 and 47 and the positions where the sections described above are disposed can be changed as appropriate.

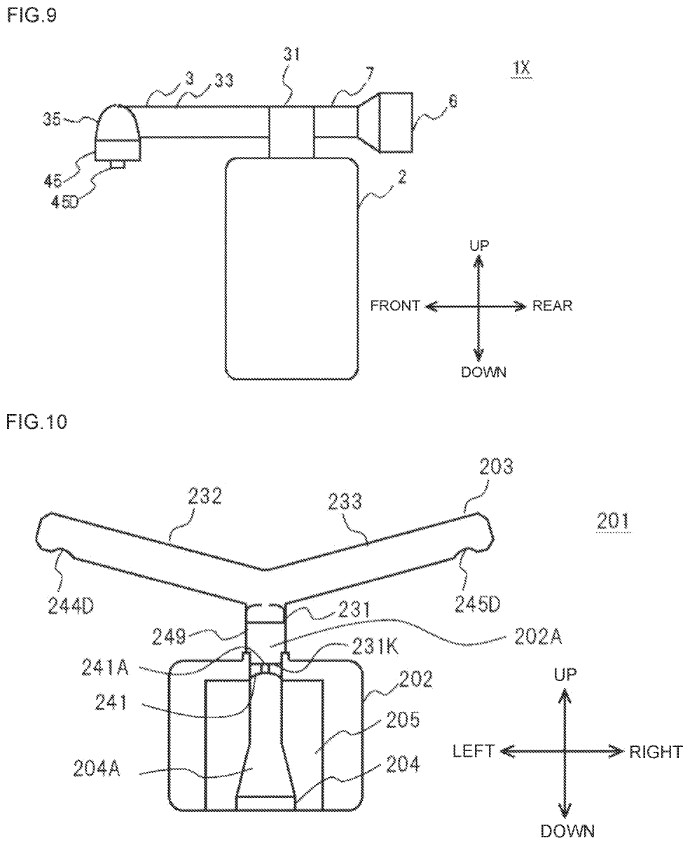

In one of the embodiments described above, the two spraying ports 44D and 45D are provided, but not necessarily in the present invention, and any number of spraying ports 44D and 45D can be provided. For example, the lateral guiding pipe 33 may extend forward from the upward guiding pipe 31, and only one spraying port 45D may be provided, as in a spraying apparatus 1X shown in FIG. 9. Although not shown, for example, three, four, or more spraying ports may be provided.

In one of the embodiments described above, the upward guiding pipe 31 is located outside the enclosure 2, but not necessarily in the present invention. For example, the upward guiding pipe 31 may be provided in the enclosure 2, the lateral guiding pipe 33 may be connected to the upward guiding pipes 31 connected to the mist generator 4 in the enclosure 2, and the lateral guiding pipe 33 may protrude laterally beyond the enclosure 2.

In one of the embodiments described above, the reservoir tank 5 as the hypochlorous acid water supplier, the mist generator 4 as the mist generator, the upward guiding pipe 31 as the upward guiding pipe, the lateral guiding pipes 32 and 33 as the lateral guiding pipe, the spraying port 45D as the spraying section, and the fan 6 as the fan constitute the spraying apparatus 1 as the spraying system according to the present invention, but not necessarily in the present invention, and a hypochlorous acid water supplier, an upward guiding pipe, a lateral guiding pipe, a spraying section, and a fan having a variety of other configurations may constitute the hypochlorous acid water spraying system according to the present invention.

### INDUSTRIAL APPLICABILITY

The present invention is applicable, for example, to a spraying apparatus that sprays hypochlorous acid water onto persons or objects.

### REFERENCE SINGS LIST

1: Spraying apparatus
2: Enclosure
3: Spraying member
4: Mist generator
5: Reservoir tank
6: Fan
7: Fan pipe
31: Upward guiding pipe
32, 33: Lateral guiding pipe
34, 35: Downward guiding pipe
41: Restrictor
42, 43: Lower blocking section
46, 47: Inclining section

1. A spraying system comprising:
a hypochlorous acid water supplier that supplies hypochlorous acid water;
a mist generator that generates mist of the hypochlorous acid water;
an upward guiding pipe that guides the mist upward;
a lateral guiding pipe connected to the upward guiding pipe and extending in a lateral direction; and
a spraying section that is provided near an end of the lateral guiding pipe and sprays the mist downward.

2. The spraying system according to claim 1, wherein a restrictor that reduces an inner diameter of the upward guiding pipe is provided in the upward guiding pipe.

3. The spraying system according to claim 2,
wherein the restrictor includes
a restricting hole formed in a center portion,
a peripheral blocking section that extends from the restricting hole toward a periphery, inclines downward, and does not allow the mist around the restricting hole to pass therethrough,
a circumferential edge hole formed at a circumferential edge of the peripheral blocking section and formed inside the upward guiding pipe.

4. The spraying system according to claim 1,
wherein the lateral guiding pipe extends outward and inclines upward by an angle ranging from 1 to 45°, and
the spray section is provided downward at a portion having ⅓ of a length of the lateral guiding pipe from an end of the lateral guiding pipe.

5. The spraying system according to claim 1,
wherein the spraying section is a spraying hole provided in the lateral guiding pipe, and
a protrusion that protrudes toward an interior of the lateral guiding pipe is provided around a circumferential edge of the spraying hole.

6. A spraying system comprising:
a hypochlorous acid water supplier that supplies hypochlorous acid water;
a mist generator that generates mist of the hypochlorous acid water;
an upward guiding pipe that guides the mist upward;
a lateral guiding pipe connected to the upward guiding pipe and extending in a lateral direction;
a fan that delivers air to the lateral guiding pipe;
a downward guiding pipe that is provided below a lower end of the lateral guiding pipe and guides the mist downward; and
a spraying section that is provided downstream from the downward guiding pipe and sprays the mist.

7. The spraying system according to claim 1, wherein the spraying section comprises a plurality of spraying sections.

8. The spraying system according to claim 7, wherein the spraying sections each include a water droplet storage capable of storing water droplets around a spraying port.

9. The spraying system according to claim 8, wherein the hypochlorous acid water supplier is a tank that stores the hypochlorous acid water.

10. The spraying system according to claim 9, wherein the hypochlorous acid water supplier is an electrolysis section that electrolyzes a supplied electrolyte solution and raw water.

11. The spraying system according to claim 1, wherein the upward guiding pipe includes an upstream section having a large diameter, a downstream section having a small diameter, and a connector that connects the upstream and downstream sections to each other.

12. The spraying system according to claim 1, wherein a lower blocking section that blocks the mist that is about to pass through a lower portion of the lateral guiding pipe is provided in the lateral guiding pipe.

13. The spraying system according to claim 1, wherein an inclination section that inclines from a downstream side toward an upstream side is provided in the lateral guiding pipe.

14. A spraying member comprising:
an upward guiding pipe that guides mist of hypochlorous acid water upward;
a lateral guiding pipe connected to the upward guiding pipe and extending in a lateral direction;
a spraying section that is provided on a lower side of the lateral guiding pipe and sprays the mist downward, and
a fan that is connected to the upward guiding pipe and delivers air into the upward guiding pipe.
